(19) United States
(12) Patent Application Publication
(10) Pub. No.: US 2026/0225986 A1
(43) Pub. Date: Aug. 6, 2026

(54) PROCESS FOR SYNTHESIS OF DIHYDROXY-DICARBOXYLIC ACID BIPHENYL COMPOUNDS

(71) Applicant: Mosaic Materials, Inc., Alameda, CA (US)

(72) Inventors: Aeri Gosselin, El Cerrito, CA (US); Michael Alexander Boreen, San Francisco, CA (US); James W. Ringer, Carmel, IN (US); Udaysinh Parmar, Vanz Gaon, Sachin, Surat, Gujarat (IN)

(21) Appl. No.: 19/042,646

(22) Filed: Jan. 31, 2025

(51) Int. Cl. C07C 51/295; C07C 51/42

(52) U.S. Cl. CPC C07C 51/295; C07C 51/42

(57) ABSTRACT

A method of making a compound represented by Formula 1 or Formula 2, the method including: combining a compound of Formula A or Formula B, respectively, with an alkali metal carbonate, and an alkali metal formate in the presence an aliphatic aromatic hydrocarbon with a boiling point from 110° C. to 180° C. to provide a reaction mixture; and conducting a reaction of the reaction mixture with an amount of carbon dioxide sufficient to maintain a reaction pressure of 50 bar or less, wherein the reaction is conducted at a temperature from 190° C. to 260° C.

### BACKGROUND

Metal-organic framework materials are compounds that include metal ions that are coordinated by bi-dentate linkers (or other multi-dentate linkers) to form crystalline structures. A group of metal-organic framework (MOF) materials for use in separations and/or catalytic applications are MOF materials that include a biphenyl structured linker is of interest. Metal-organic framework materials based on such linkers can potentially be used for selective adsorption of carbon, e.g., CO₂ or CO, in industrial fluid streams, carbon emission from fossil fuel extraction or combustion processes as well as carbon removal from the atmosphere, e.g., the air surrounding urban areas.

One of the difficulties in the commercial application of MOF materials is that biphenyl structured linkers can be relatively expensive to make using existing methods, which, of course, raises the cost for the MOFs. In some instances, conventional methods for the synthesis of biphenyl structured linkers can be difficult to scale up for production. Additionally, some synthetic methods can require high cost starting materials, harsh reaction agents, or high reaction pressures.

In the Kolbe-Schmitt (KS) reaction, 4,4′-dihydroxybiphenyl is subjected to high pressure and temperature conditions using expensive and non-environmentally friendly chemical reagents such as N,N-dimethylformamide (DMF) or trichlorobenzene due to the low solubility of the reagents used in the making of 4,4′-dihydroxy-biphenyl-3,3′-dicarboxylic acid (H₄DOBPDC).

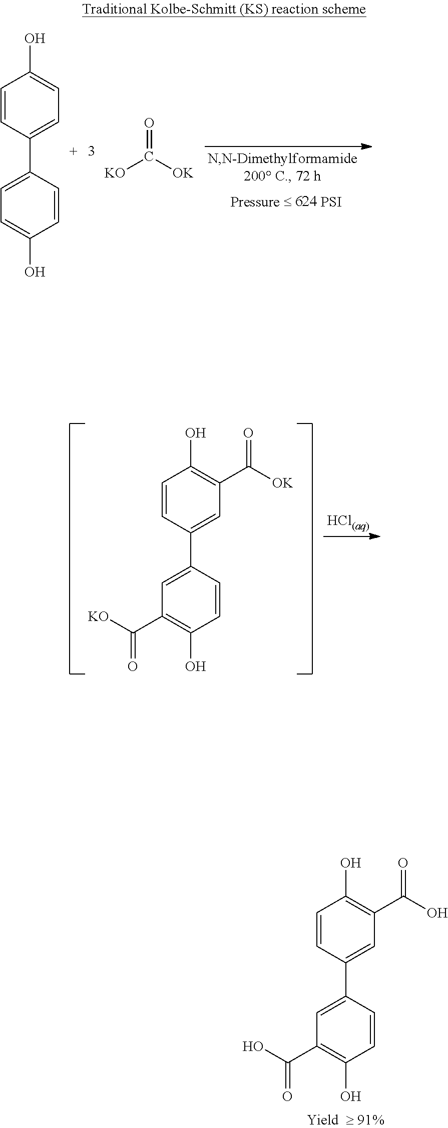

U.S. Patent Pub. 2021/0230092 describes a method for forming H₄DOBPDC based on a reaction of 4,4′-biphenol in an amide solvent (such as dimethylformamide) in the presence of a base. The use of dimethylformamide improves the solubility of the reactant 4,4′-biphenol. The reaction is described as beneficial by providing a synthesis route that can be performed at lower pressures and lower temperatures than conventional synthesis based on the traditional (KS) reaction.

WO2023189499 discloses a process to prepare H₄DOBPDC in high yield by a process including reacting an alkali salt of 4,4′-dihydroxybiphenyl with carbon dioxide in a light oil in the presence of a metal salt of acetic acid. The reaction is conducted at temperatures above 150° C. and a pressure of 1 to 50 bar.

WO2023/147354 describes a method for making H₄DOBPDC from 4,4′-biphenol in the presence of CO₂ and a base and under temperature and pressure conditions for generating supercritical CO₂. Accordingly, in theory, it would be possible to conduct the carboxylation reaction in the absence of any solvent. The resulting product is then dissolved in water and unreacted reagents are filtered off. However, the supercritical feature of the process introduces high temperatures (exceeding 280° C.). Moreover, to increase production yield and/or product purity, the addition of a solvent including 50 vol % or more of dioxane is generally required.

### SUMMARY

In one aspect, a method of making a compound represented by Formula 1 is described. The method comprising:

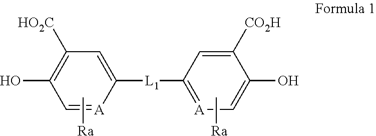

combining a compound of Formula A with a metal carbonate, and a metal formate in the presence an aliphatic aromatic hydrocarbon with a boiling point from 110° C. to 180° C. to provide a reaction mixture,

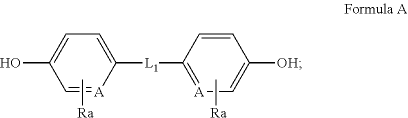

and
conducting a reaction of the reaction mixture with an amount of carbon dioxide sufficient to maintain a reaction pressure of 15 to 50 bar, wherein the reaction is conducted at a temperature from 190° C. to 260° C.;
wherein in Formula 1 and Formula A:
each A is independently C or N;
L₁ is a single bond, O, S, CH₂, CF₂, C(CH₃)₂, C(CF₃)₂, a C₂-C₃ alkylene, a C₄-C₁₀ arylene, or a combination thereof; and
each Rₐ is independently H, F, CN, C₁-C₄ alkyl, or C₁-C₄ alkoxy.

In an aspect, each A is C and each Rₐ is H.

In an aspect, a mole ratio of metal carbonate: metal formate is 1:4 to 4:1, or 1:2.5 to 2.5:1, per mole of the compound of Formula A, where M is an alkali metal.

In an aspect, the aromatic solvent is o-xylene, m-xylene, p-xylene, hemimellitene, pseudocumene, mesitylene, ethylbenzene, n-propylbenzene, cumene, iso-butylbenzene, sec-butylbenzene, tert-butylbenzene, p-cymene, or a combination thereof. In another aspect, the aromatic solvent is o-xylene, m-xylene, p-xylene, mesitylene, ethylbenzene, cumene, or a combination thereof.

In an aspect, the amount of carbon dioxide is sufficient to maintain the reaction pressure from 15 bar to 40 bar, or from 15 bar to 30 bar.

In an aspect, the reaction is conducted for 100 hours or less to provide a product yield of 55% or greater. In another aspect, the reaction is conducted for 80 hours or less to provide a product yield of 65% or greater.

In an aspect, the method further comprises washing the compound of Formula 1 in a C₁-C₄ alcohol to remove reaction product impurities.

In an aspect, the compound of Formula 1 is synthesized with 90% purity or greater.

In an aspect, the compound of Formula A is 4,4′-biphenol, the mole ratio of metal carbonate: metal formate is 1:4 to 4:1, per mole of the compound of Formula A, the aromatic solvent is o-xylene, the reaction temperature is from 195° C. to 240° C., and the amount of carbon dioxide is sufficient to maintain the reaction pressure from 15 bar to 40 bar.

In another aspect, a method of making a compound represented by Formula 2 is described. The method comprising:

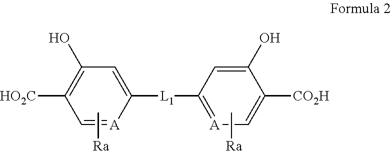

combining a compound of Formula B with a metal carbonate, and a metal formate in the presence an aliphatic aromatic hydrocarbon with a boiling point from 110° C. to 180° C. to provide a reaction mixture,

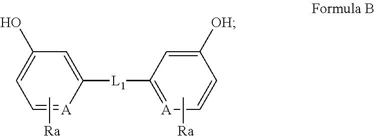

and
conducting a reaction of the reaction mixture with an amount of carbon dioxide sufficient to maintain a reaction pressure of 15 to 50 bar, wherein the reaction is conducted at a temperature from 190° C. to 260° C.;
wherein in Formula 2 and Formula B:
each A is independently C or N;
L₁ is a single bond, O, S, CH₂, CF₂, C(CH₃)₂, C(CF₃)₂, a C₂-C₃ alkylene, a C₄-C₁₀ arylene, or a combination thereof; and
each Rₐ is independently H, F, CN, C₁-C₄ alkyl, or C₁-C₄ alkoxy.

In an aspect, each A is C and each Rₐ is H.

In an aspect, a mole ratio of metal carbonate: metal formate is 1:4 to 4:1, or 1:2.5 to 2.5:1, per mole of the compound of Formula B, where M is an alkali metal.

In an aspect, the aromatic solvent is o-xylene, m-xylene, p-xylene, hemimellitene, pseudocumene, mesitylene, ethylbenzene, n-propylbenzene, cumene, iso-butylbenzene, sec-butylbenzene, tert-butylbenzene, p-cymene, or a combination thereof, and the amount of carbon dioxide is sufficient to maintain the reaction pressure from 15 bar to 40 bar.

In an aspect, the reaction is conducted for 80 hours or less to provide a product yield of 65% or greater.

In an aspect, the method further comprises washing the compound of Formula 2 in a C₁-C₄ alcohol to remove reaction product impurities.

### DETAILED DESCRIPTION

In various aspects, methods are provided for the carboxylation of aromatic alcohols represented by Formula A or Formula B infra. The aromatic alcohol reagent can correspond to a C₂-symmetrical or asymmetrical compound that includes at least two hydroxyl (—OH) groups attached to ring carbons of at least two different aromatic rings within the aromatic alcohol. The aromatic alcohol reagent can be selected so that the relative ring position of the alcohol groups in the reagent alcohol matches the ring position of the hydroxyl group in the target compound. The aromatic alcohol requires that at least one “ortho” position on the aromatic ring relative to each hydroxyl group be available for carboxylation, i.e., the addition of a CO₂ to form a carboxylate group at a ring carbon adjacent to the hydroxy group. In some aspects, some benefit may be provided by having both “ortho” (adjacent) positions available. One example of an aromatic alcohol reagent is 4,4′-biphenol having both ortho positions available for carboxylation to form 4,4′-dihydroxybiphenyl-3,3′-dicarboxylic acid (H₄DOBPDC).

A detailed description of one or more embodiments including methods of preparation are presented herein by way of exemplification.

In an aspect, a method of making a compound represented by Formula 1 is described. The method of preparation includes:

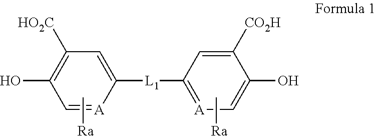

combining a compound of Formula A with an alkali metal carbonate, and an alkali metal formate in the presence an aliphatic aromatic hydrocarbon with a boiling point from 110° C. to 180° C. to provide a reaction mixture,

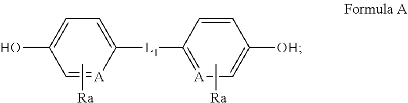

and
conducting a reaction of the reaction mixture with an amount of carbon dioxide sufficient to maintain a reaction pressure of 15 bar to 50 bar, wherein the reaction is conducted at a temperature from 190° C. to 260° C.;
wherein in Formula 1 and Formula A:
each A is independently C or N;
L₁ is a single bond, O, S, CH₂, CF₂, C(CH₃)₂, C(CF₃)₂, a C₂-C₃ alkylene, a C₄-C₁₀ arylene, or a combination thereof; and
each Rₐ is independently H, F, CN, C₁-C₄ alkyl, or C₁-C₄ alkoxy.

In an aspect, a method of making a compound represented by Formula 2 is described. The method of preparation includes:

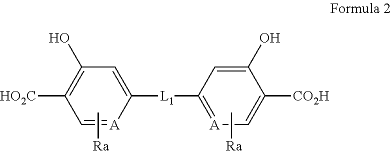

combining a compound of Formula B with an alkali metal carbonate, and an alkali metal formate in the presence an aliphatic aromatic hydrocarbon with a boiling point from 110° C. to 180° C. to provide a reaction mixture,

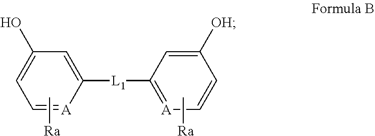

and
conducting a reaction of the reaction mixture with an amount of carbon dioxide sufficient to maintain a reaction pressure of 15 bar to 50 bar, wherein the reaction is conducted at a temperature from 190° C. to 260° C.;
wherein in Formula 2 and Formula B, each A, L₁ and each Rₐ is defined above.

In an aspect, in Formula 1 or Formula 2, each A is C and each Rₐ is H.

In an aspect, the formate and carbonate metal salts are independently in the form of sodium or potassium salts with the potassium salts being preferred. The mole equivalents of each of the alkali metal salts in relation to one mole equivalent of compound of Formula A or Formula B, e.g., biphenol, are not too important with the exception that the presence of both the alkali salts of formate (MHC(O)O) and carbonate (M₂C(O)O₂), e.g., KHC(O)O and K₂C(O)O₂, need to be present in the reaction mixture for carboxylation to proceed with acceptable product yields. As defined above, M is an alkali metal.

In an aspect, a mole ratio of M₂C(O)O₂: MHC(O)O is 4:1 to 4:1, or 1:2.5 to 2.5:1, per mole of the compound of Formula A or Formula B. In another aspect, a mole ratio of M₂C(O)O₂: MHC(O)O is 1:2 to 2:1, per mole of the compound of Formula A.

One objective of the preparation research was to determine whether the carboxylation reaction described could produce compounds of Formula 1 from compounds of Formula A, or compounds of Formula 2 from compounds of Formula B, without the need to use reaction solvents such as aromatic chlorinated solvents or DMF, which are considered less preferable in commercial-scale processes. Although such solvents may not be preferred in the carboxylation reaction described, such solvents may still be used in the processes. For example, the described carboxylation reaction performs equally well in aromatic chlorinated solvents, e.g., the different isomers of dichlorobenzene as well as mixtures thereof.

In this regard, the described carboxylation processes provide an option, e.g., a commercially-viable option, to conduct the carboxylation reaction, i.e., as a modified KS process, in the presence of an aliphatic aromatic hydrocarbon with a boiling point from 110° C. to 180° C.

In an aspect, the aliphatic aromatic solvent is o-xylene, m-xylene, p-xylene, hemimellitene, pseudocumene, mesitylene, ethylbenzene, n-propylbenzene, cumene, iso-butylbenzene, sec-butylbenzene, tert-butylbenzene, p-cymene, or a combination thereof. A select list of aliphatic aromatic solvents is o-xylene, m-xylene, p-xylene, mesitylene, ethylbenzene, cumene, or a combination thereof.

In an aspect, the reaction temperature for the carboxylation reaction is from 190° C. to 240° C., e.g., from 195° C. to 225° C., and the amount of carbon dioxide present in the reaction is sufficient to maintain the reaction pressure from 15 bar to 50 bar, e.g., from 15 bar to 40 bar.

In an aspect, the reaction temperature for the carboxylation reaction is from 200° C. to 220° C., and the amount of carbon dioxide is sufficient to maintain the reaction pressure from 15 bar to 30 bar.

In an aspect, if L₁ includes a C₄-C₁₀ arylene linker, the C₄-C₁₀ arylene linker may be represented by Formula C1 or Formula C2:

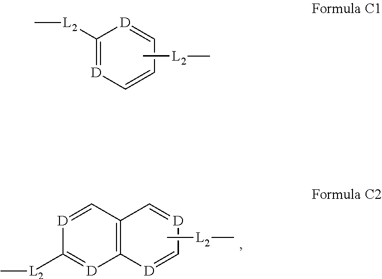

wherein each D is independently C or N, and
each L₂ is a single bond, O, CH₂, CF₂, C(CH₃)₂, C(CF₃)₂.

In an aspect, if L₁ includes a C₄-C₁₀ arylene linker, represented by Formula C1 or Formula C2, each D is C and L₂ is independently a single bond or CH₂. In another aspect, each aromatic ring may include one N ring atom and L₂ is independently a single bond or CH₂.

In accordance with an aspect, the improved batch-wise process for the synthesis of 4,4′-dihydroxybiphenyl-3,3′-dicarboxylic acid (HADOBPDC) is a modified version of the KS reaction process. The modified KS process utilizes less costly reagents and readily available starting materials. Additionally, the modified KS process uses an alkali metal carbonate salt as a source of CO₂ in the reaction. Moreover, we found that the addition of an additional salt, i.e., the alkali metal formate and the presence of CO₂ at sufficient pressure, can lead to a relatively high-quality material with relatively short reaction times and lower process pressures, e.g., about 20 to 30 bar compared to 43 bar in the present KS process. Moreover, the large-scale production yields of the modified KS process can exceed 55%, e.g., large-scale production yields can exceed 65% or exceed 70%. Lastly, the cost for producing large scale amounts of H₄DOBPDC is significantly less than the present KS process.

In accordance with an embodiment, an exemplary modified KS process is represented in Reaction Scheme 1.

In an aspect, and in accordance with Reaction Scheme 1, the compound of Formula 1 is 4,4′-biphenol, the mole ratio of K₂C(O)O₂: KHC(O)O is 1:1 to 3:1, the aromatic solvent is o-xylene, the reaction temperature is from 195° C. to 260° C., e.g., from 200° C. to 240° C. or from 200° C. to 230° C., and the amount of carbon dioxide is sufficient to maintain the reaction pressure from 15 bar to 40 bar, or from 15 bar to 30 bar.

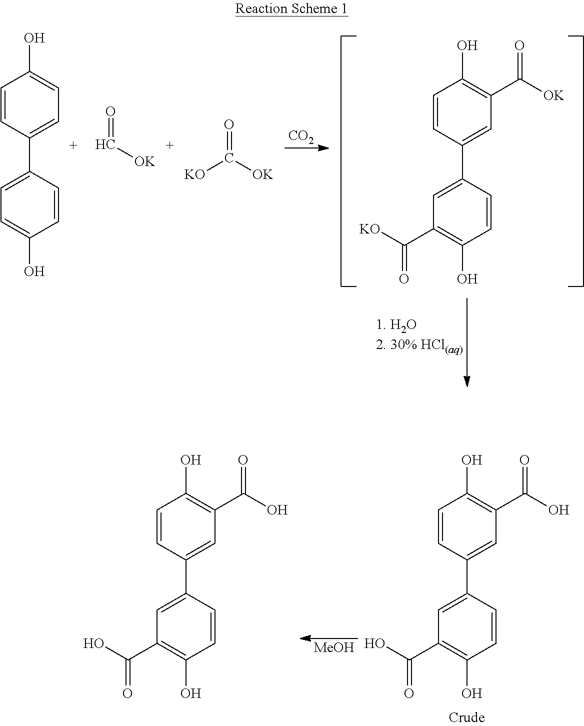

The modified KS process described herein may be conducted for 100 hours or less, e.g., 80 hours or less or 60 hours or less, within the above range of reaction temperatures and pressures to provide a crude product yield of 55% or greater, e.g. 65% or greater, and in many large-scale commercial batch productions the crude product yields may exceed 75% or 80%. Moreover, the modified KS process can provide a crude product with relatively low side reactions and other impurities. In fact, the post-reaction purification process is essentially used to remove any remaining alkali metal and unreacted alkali metal salts from the crude product.

The purification methods for removing side products and impurities in conventional KS carboxylation processes is generally well known. For example, the use of C₁-C₄ alcohols such as ethanol or isopropanol as well as other wash solvents such as acetone, ethyl acetate can be used to remove any remaining alkali metal and unreacted alkali metal salts from the crude product. Of course, any mixture of common wash solvents used to remove impurities from substituted aromatic compounds, e.g., biphenyl compounds, can be used as well.

In an aspect, the crude reaction product including the compounds of Formula 1 or Formula 2 may be washed with several portions of methanol to remove the above impurities and to provide compounds of Formula 1 of 90% purity or greater, e.g., 93% purity or greater.

The use of the terms “a” and “an” and “the” and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another. The terms “about”, “substantially” and “generally” are intended to include the degree of error associated with measurement of the particular quantity based upon the equipment available at the time of filing the application. For example, “about” and/or “substantially” and/or “generally” can include a range of +8% of a given value.

### Examples

Example 1:4,4′-Biphenol (1.25 kg, 6.71 mol, 1 equiv), potassium carbonate (1.39 kg, 10.07 mol, 1.5 equiv), and potassium formate (0.85 kg, 10.070 mol, 1.5 equiv), were added to a 25-liter autoclave with o-xylene (10 L). Carbon dioxide (~29 psi) was added to the reaction mixture. Upon heating to 225° C., the pressure increased to approximately ~155 psi (10.7 bar). The temperature was maintained at 225° C., and additional CO₂ was introduced to achieve a pressure of 290 psi (20 bar), which was sustained for 50 hours. The pressure was maintained at 20 bar by continuously adding CO₂ gas during the reaction.

The mixture was cooled to room temperature and water (12.5 L) was added. The mixture was stirred for 30 minutes to obtain a free-flowing slurry. A sample was taken for HPLC analysis. The product light pink solids were suspended in water, and the pH was adjusted with aqueous 30% HCl solution to a pH of 2 while stirring at 30° C. A color change from light pink to light purple and then to a light cream at pH of 2 was observed. The suspension was stirred at 35° C. for 2 hours. The solid was filtered and washed with water (40 liters, 10×4 liters) to neutralize the pH (remove excess HCl). The wet cake was dried at 60° C. for 18 hours in a vacuum oven to yield the crude H₄DOBPDC with % purity (HPLC) of 97%, crude product yield, 92%.

Example 5; The process of Example 1 was repeated except the mole equivalents of potassium carbonate to potassium formate was 2:1 and the reaction time increased to 60 hours. The wet cake was dried at 60° C. for 18 hours in a vacuum oven to yield the crude H₄DOBPDC, yield, 93%.

Example 6: The process of Example 3 was repeated except the mole equivalents of potassium carbonate to potassium formate was 1:4 and the reaction time increased to 60 hours. The wet cake was dried at 60° C. for 18 hours in a vacuum oven to yield the crude H₄DOBPDC, yield, 84%.

Purification of Example 1: The crude H₄DOBPDC (1.6 kg) was suspended in methanol (16.3 L) for purification. The mixture was heated to 60° C. for 1 hour and then hot-filtered at 60° C. The wet cake was washed with 500 mL of methanol and suction-dried for 2 hours. The wet cake was further dried at 50° C. for 4 hours in a vacuum oven to yield the pure H₄DOBPDC, 69% yield.

Comparative Example 1: The process of Example 3 was repeated except benzyl alcohol was used as the reaction solvent, and the reaction time was 57 hours. The wet cake was dried at 60° C. for 18 hours in a vacuum oven to yield the crude H₄DOBPDC, yield 27%.

Comparative Example 2: The process of Example 1 was repeated except benzyl alcohol was used as the reaction solvent, and the reaction time was 8 hours. The wet cake was dried at 60° C. for 18 hours in a vacuum oven to yield the crude H₄DOBPDC, yield 37%.

Comparative Example 3: The process of Example 1 was repeated except potassium carbonate was added in 2.0 equivalents per mole of 4,4′-biphenol, and no potassium formate was added. The yield of H₄DOBPDC was less than 1%.

Comparative Example 4: The process of Example 3 was repeated except potassium formate was added in 2.0 equivalents per mole of 4,4′-biphenol, and no potassium carbonate was added. The yield of H₄DOBPDC was 13%.

Comparative Example 5: The process of Example 3 was repeated except the pressure was maintained at 9.2 bar. The reaction was stopped after 47 hours with a H₄DOBPDC yield of 15%.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A method of making a compound represented by Formula 1, the method comprising: 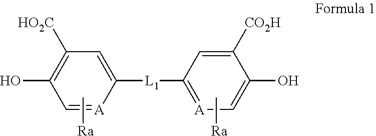
combining a compound of Formula A with a metal carbonate, and a metal formate in the presence an aliphatic aromatic hydrocarbon with a boiling point from 110° C. to 180° C. to provide a reaction mixture, 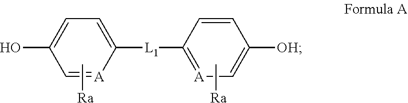
and
conducting a reaction of the reaction mixture with an amount of carbon dioxide sufficient to maintain a reaction pressure of 15 to 50 bar, wherein the reaction is conducted at a temperature from 190° C. to 260° C.;
wherein in Formula 1 and Formula A:
each A is independently C or N;
L₁ is a single bond, O, S, CH₂, CF₂, C(CH₃)₂, C(CF₃)₂, a C₂-C₃ alkylene, a C₄-C₁₀ arylene, or a combination thereof; and
each Rₐ is independently H, F, CN, C₁-C₄ alkyl, or C₁-C₄ alkoxy.

2. The method of claim 1, wherein each A is C and each Rₐ is H.

3. The method of claim 1, wherein a mole ratio of metal carbonate: metal formate is 1:4 to 4:1, per mole of the compound of Formula A, where M is an alkali metal.

4. The method of claim 1, wherein a mole ratio of metal carbonate: metal formate is 1:2.5 to 2.5:1, per mole of the compound of Formula A, where M is an alkali metal.

5. The method of claim 1, wherein the aromatic solvent is o-xylene, m-xylene, p-xylene, hemimellitene, pseudocumene, mesitylene, ethylbenzene, n-propylbenzene, cumene, iso-butylbenzene, sec-butylbenzene, tert-butylbenzene, p-cymene, or a combination thereof.

6. The method of claim 5, wherein the aromatic solvent is o-xylene, m-xylene, p-xylene, mesitylene, ethylbenzene, cumene, or a combination thereof.

7. The method of claim 1, wherein the amount of carbon dioxide is sufficient to maintain the reaction pressure from 15 bar to 40 bar.

8. The method of claim 5, wherein the reaction temperature is from 200° C. to 220° C., and the amount of carbon dioxide is sufficient to maintain the reaction pressure from 15 bar to 30 bar.

9. The method of claim 1, wherein the reaction is conducted for 70 hours or less to provide a product yield of 55% or greater.

10. The method of claim 8, wherein the reaction is conducted for 60 hours or less to provide a product yield of 65% or greater.

11. The method of claim 1, further comprising washing the compound of Formula 1 in a C₁-C₄ alcohol to remove reaction product impurities.

12. The method of claim 11, wherein the compound of Formula 1 is of 90% purity or greater.

13. The method of claim 1, wherein the compound of Formula A is 4,4′-biphenol, the mole ratio of metal carbonate: metal formate is 1:4 to 4:1, per mole of the compound of Formula A, the aromatic solvent is o-xylene, the reaction temperature is from 195° C. to 240° C., and the amount of carbon dioxide is sufficient to maintain the reaction pressure from 15 bar to 40 bar.

14. A method of making a compound represented by Formula 2, the method comprising: 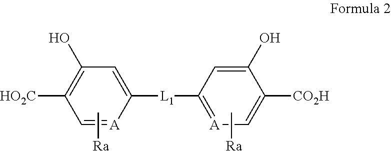
combining a compound of Formula B with a metal carbonate, and a metal formate in the presence an aliphatic aromatic hydrocarbon with a boiling point from 110° C. to 180° C. to provide a reaction mixture, 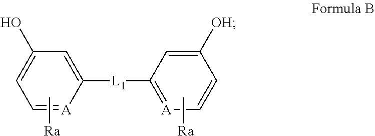
and
conducting a reaction of the reaction mixture with an amount of carbon dioxide sufficient to maintain a reaction pressure of 15 to 50 bar, wherein the reaction is conducted at a temperature from 190° C. to 260° C.;
wherein in Formula 1 and Formula B:
each A is independently C or N;
L₁ is a single bond, O, S, CH₂, CF₂, C(CH₃)₂, C(CF₃)₂, a C₂-C₃ alkylene, a C₄-C₁₀ arylene, or a combination thereof; and
each Rₐ is independently H, F, CN, C₁-C₄ alkyl, or C₁-C₄ alkoxy.

15. The method of claim 14, wherein each A is C and each Rₐ is H.

16. The method of claim 14, wherein a mole ratio of metal carbonate: metal formate is 1:4 to 4:1, per mole of the compound of Formula B, where M is an alkali metal.

17. The method of claim 14, wherein a mole ratio of metal carbonate: metal formate is 1:2.5 to 2.5:1, per mole of the compound of Formula B, where M is an alkali metal.

18. The method of claim 14, wherein the aromatic solvent is o-xylene, m-xylene, p-xylene, hemimellitene, pseudocumene, mesitylene, ethylbenzene, n-propylbenzene, cumene, iso-butylbenzene, sec-butylbenzene, tert-butylbenzene, p-cymene, or a combination thereof, and the amount of carbon dioxide is sufficient to maintain the reaction pressure from 15 bar to 40 bar.

19. The method of claim 14, wherein the reaction is conducted for 80 hours or less to provide a product yield of 65% or greater.

20. The method of claim 14, further comprising washing the compound of Formula 1 in a C₁-C₄ alcohol to remove reaction product impurities.
